(19) United States
(12) Patent Application Publication
(10) Pub. No.: US 2017/0131083 A1
(43) Pub. Date: May 11, 2017

(54) TOMOGRAPHY APPARATUS BASED ON LOW COHERENCE INTERFEROMETER

(71) Applicant: IUCF-HYU (INDUSTRY-UNIVERSITY COOPERATION FOUNDATION HANYANG UNIVERSITY), Seoul (KR)

(72) Inventors: Younggeun HAN, Seoul (KR); Sunduck KIM, Gunsan-si (KR)

(73) Assignee: IUCF-HYU (INDUSTRY-UNIVERSITY COOPERATION FOUNDATION HANYANG UNIVERSITY), Seoul (KR)

(21) Appl. No.: 15/323,223

(22) PCT Filed: Jul. 3, 2015
(86) PCT No.: PCT/KR2015/006888

§ 371 Date: Dec. 30, 2016

(30) Foreign Application Priority Data
Jul. 3, 2014 (KR) 10-2014-0083016

(51) Int. Cl. G01B 9/02; A61B 6/03

(52) U.S. Cl. CPC G01B 9/02091; A61B 6/03; G01B 9/02015

(57) ABSTRACT

A tomography apparatus based on a low coherence interferometer according to embodiments of the inventive concepts may include a plurality of wavelength-tunable lasers arranged in parallel, and an optical coupling unit interleaving pulses sequentially outputted from the plurality of wavelength-tunable lasers to increase a wavelength tuning speed of the wavelength-tunable lasers by N times where ‘N’ corresponds to the number of the wavelength-tunable lasers. According to embodiments of the inventive concepts, the tomography apparatus may rapidly increase the wavelength tuning speed by applying the interleaving technique to obtain accurate tomographic image information, and thus the tomography apparatus can be widely used in medical fields (e.g., medical engineering and biomedical engineering), an aerospace field, a spectroscopy field, and a sensor field.

### CROSS-REFERENCE TO RELATED APPLICATION

This application is a continuation of pending International Application No. PCT/KR2015/006888, which was filed on Jul. 3, 2015 and claims priority to Korean Patent Application No. 10-2014-0083016, filed on Jul. 3, 2014, in the Korean Intellectual Property Office, the disclosures of which are hereby incorporated by reference in their entireties.

### BACKGROUND

1. Field

The present disclosure relates to a tomography apparatus based on a low coherence interferometer. More particularly, the present disclosure relates to a tomography apparatus based on a low coherence interferometer, which can rapidly increase a wavelength tuning speed by applying an interleaving technique to obtain accurate tomographic image information and thus can be widely applied to medical fields (e.g., medical engineering and biomedical engineering), an aerospace field, a spectroscopy field, and a sensor field.

2. Description of the Related Art

A low coherence interferometer is a next-generation technique that combines biological and medical fields with an optical technique. Since the low coherence interferometer observes diseases of the eyes, skin and internal organs in real time without damage, it is being in the spotlight. In particular, the low coherence interferometer can accurately measure a surface of a bending skin and can be applied to an essential inspection instrument for telemedicine if it can rapidly tune or vary a wavelength.

A general low coherence interferometer uses a conventional wavelength-tunable laser. However, the conventional wavelength-tunable later has a limited driving speed, and thus the general low coherence interferometer does not obtain an image of which a speed is faster than a tuning speed of the wavelength-tunable laser. In other words, a wavelength tuning speed of the conventional wavelength-tunable laser is determined by the driving speed thereof, and thus the conventional wavelength-tunable laser does not rapidly tune or vary a wavelength.

Therefore, it is required to develop a low coherence interferometer-based apparatus having a new structure capable of rapidly tuning or varying a wavelength.

### SUMMARY

Embodiments of the inventive concepts provide a tomography apparatus based on a low coherence interferometer, which can rapidly increase a wavelength tuning speed by applying an interleaving technique to obtain accurate tomographic image information and thus can be widely applied to medical fields (e.g., medical engineering and biomedical engineering), an aerospace field, a spectroscopy field, and a sensor field.

In an aspect, a tomography apparatus based on a low coherence interferometer may include a plurality of wavelength-tunable lasers arranged in parallel, and an optical coupling unit interleaving pulses sequentially outputted from the plurality of wavelength-tunable lasers to increase a wavelength tuning speed of the wavelength-tunable lasers by N times where ‘N’ corresponds to the number of the wavelength-tunable lasers. By these elements, the tomography apparatus may rapidly increase the wavelength tuning speed by applying the interleaving technique to obtain the accurate tomographic image information, and thus the tomography apparatus can be widely used in medical fields (e.g., medical engineering and biomedical engineering), an aerospace field, a spectroscopy field, and a sensor field.

In some embodiments, the number of the plurality of wavelength-tunable lasers may be N where ‘N’ is a natural number, and the wavelength-tunable lasers may have the same center wavelength and the same wavelength tuning range. A speed of the wavelength-tunable lasers may be increased by N times by interleaving and coupling the pulses of the plurality of wavelength-tunable lasers, each of which has a pulse width corresponding to 1/N of a repetition period of the plurality of wavelength-tunable lasers.

In some embodiments, the number of the plurality of wavelength-tunable lasers may be N where ‘N’ is a natural number, and center wavelengths and wavelength tuning ranges of the wavelength-tunable lasers may sequentially increase. A wavelength tuning bandwidth N times wider than the maximum wavelength tuning bandwidth of the wavelength-tunable lasers may be obtained by interleaving and coupling the pulses of the plurality of wavelength-tunable lasers, each of which has a pulse width corresponding to 1/N of a repetition period of the plurality of wavelength-tunable lasers.

In some embodiments, the tomography apparatus may further include a splitting unit connected to the optical coupling unit to split pulses optically coupled by the optical coupling unit into a sample stage and a reference stage, and an optical detector obtaining an interference signal from pulses transmitted through the splitting unit via the sample stage and the reference stage.

In some embodiments, the splitting unit may be a beam splitter splitting a beam or an optical coupler based on an optical waveguide.

In some embodiments, the tomography apparatus may further include a plurality of mirrors respectively provided at rears of the plurality of wavelength-tunable lasers to parallel the pulses generated from the plurality of wavelength-tunable lasers, and a beam reduction unit reducing a beam of the pulses incident in parallel by the plurality of mirrors. The optical coupling unit may be provided based on an optical waveguide to optically couple the pulses beam-reduced by the beam reduction unit.

In some embodiments, the plurality of wavelength-tunable lasers may be respectively connected to optical waveguides, each of which has a shape becoming narrower in a guiding direction. The optical coupling unit may be provided in an optical waveguide type having a core such that cores of the optical waveguides connected to the plurality of wavelength-tunable lasers may be connected to the core of the optical coupling unit to interleave the pulses sequentially outputted from the plurality of wavelength-tunable lasers.

In some embodiments, the optical coupling unit may be provided in plurality. The pulses sequentially generated from a first wavelength-tunable laser and a second wavelength-tunable laser of the plurality of wavelength-tunable lasers may be optically coupled to each other by a first optical coupling unit of the optical coupling units. A pulse generated by the first optical coupling unit and the pulse generated from a third wavelength-tunable laser of the plurality of wavelength-tunable lasers may be optically coupled to each other by a second optical coupling unit of the optical coupling units. By this optical coupling method, optical coupling processes may be sequentially performed up to the last wavelength-tunable laser of the wavelength-tunable lasers.

In some embodiments, the optical coupling unit may be any one of an array waveguide grating and a 1×N optical coupler.

In some embodiments, each of the plurality of wavelength-tunable lasers may be connected to the optical coupling unit through an optical waveguide. The optical waveguide may be any one of an optical fiber, a LiNbO₃ waveguide, an ion exchanged glass coupler, a SiO₂/Si waveguide, and a polymer waveguide.

In some embodiments, each of the wavelength-tunable lasers may be any one of a Fourier domain mode locking laser based on a fiber Fabry-Perot filter, a Fourier domain mode locking laser based on a grating and a galvo mirror, a Fourier domain mode locking laser based on a grating and a polygon mirror, a fiber-based wavelength-tunable laser of a distributed control-based wavelength-tunable laser, a wavelength-tunable laser based on a polymer waveguide grating, and a wavelength-tunable laser based on MEMS VCSEL.

### BRIEF DESCRIPTION OF THE DRAWINGS

FIG. 1 is a schematic diagram illustrating a tomography apparatus based on a low coherence interferometer, according to a first embodiment of the inventive concepts.

FIG. 2 is a diagram illustrating a process of obtaining an N times faster wavelength tuning speed from wavelength-tunable lasers illustrated in FIG. 1.

FIG. 3 is a diagram illustrating a process of obtaining an N times wider wavelength bandwidth from the wavelength-tunable lasers illustrated in FIG. 1.

FIG. 4 is a schematic diagram illustrating a tomography apparatus based on a low coherence interferometer, according to a second embodiment of the inventive concepts.

FIG. 5 is a schematic diagram illustrating a portion of a tomography apparatus based on a low coherence interferometer, according to a third embodiment of the inventive concepts.

FIG. 6 is a schematic diagram illustrating a portion of a tomography apparatus based on a low coherence interferometer, according to a fourth embodiment of the inventive concepts.

FIG. 7 is a schematic diagram illustrating a portion of a tomography apparatus based on a low coherence interferometer, according to a fifth embodiment of the inventive concepts.

### DETAILED DESCRIPTION OF THE EMBODIMENTS

The inventive concepts will now be described more fully hereinafter with reference to the accompanying drawings, in which exemplary embodiments of the inventive concepts are shown. The following description is one of many aspects of the claimed inventive concepts, and the following description corresponds to part of detailed descriptions of the inventive concepts.

However, in explanation of the inventive concepts, the descriptions to elements and functions of related arts may be omitted for clarity.

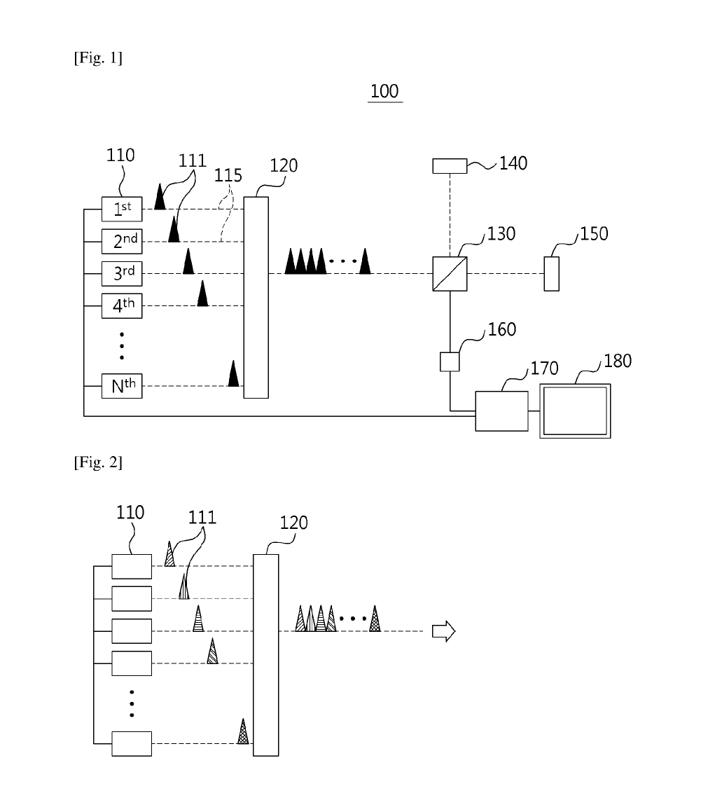

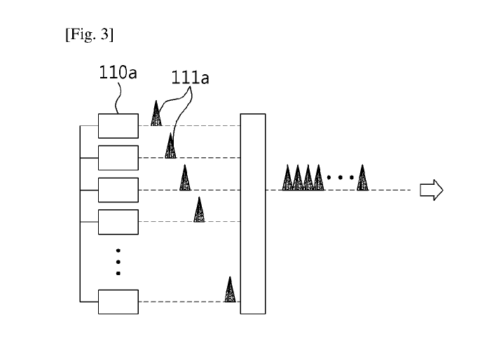

FIG. 1 is a schematic diagram illustrating a tomography apparatus based on a low coherence interferometer, according to a first embodiment of the inventive concepts. FIG. 2 is a diagram illustrating a process of obtaining an N times faster wavelength tuning speed from wavelength-tunable lasers illustrated in FIG. 1, and FIG. 3 is a diagram illustrating a process of obtaining an N times wider wavelength bandwidth from the wavelength-tunable lasers illustrated in FIG. 1.

Referring to FIG. 1, according to a first embodiment of the inventive concepts, a tomography apparatus 100 based on a low coherence interferometer (hereinafter, referred to as ‘a low coherence interferometer-based tomography apparatus 100’) may include a plurality of wavelength-tunable lasers 110 arranged in parallel, an optical coupling unit 120 interleaving pulses 111 sequentially outputted from the plurality of wavelength-tunable lasers 110 to increase a wavelength tuning speed of the wavelength-tunable lasers 110 by N times where ‘N’ corresponds to the number of the wavelength-tunable lasers 110, a beam splitter 130 connected to the optical coupling unit 120 to beam-split the pules optically coupled by the optical coupling unit 120 into a sample stage 150 and a reference stage 140, an optical detector 160 obtaining an interference signal from pulses transmitted through the beam splitter 130 via the sample stage 150 and the reference stage 140, a signal processing unit 170 analyzing the obtained interference signal, and a display unit 180 displaying signal-processed information.

Each of the elements will be described. First, the plurality of wavelength-tunable lasers 110 may sequentially output the pulses 111. Referring to FIG. 1, the topmost wavelength-tunable laser 110 completes the output of the pulse 111, and at the same time, the next wavelength-tunable laser 110 outputs the pulse 111.

As illustrated in FIG. 2, the pulses 111 generated from the plurality of (N) wavelength-tunable lasers 110 have the same center wavelength and the same wavelength tuning range, and the speed of the wavelength-tunable lasers 110 may be increased by N times by interleaving and coupling the pulses 111 of the wavelength-tunable lasers 110, each of which has a pulse width corresponding to 1/N of a repetition period of the plurality of wavelength-tunable lasers 110.

Meanwhile, as illustrated in FIG. 3, center wavelengths and wavelength tuning ranges of pulses 111a generated from a plurality of wavelength-tunable lasers 110a sequentially increase, and a wavelength tuning bandwidth N times wider than the maximum wavelength tuning bandwidth of the wavelength-tunable lasers 110a may be obtained by interleaving and coupling the pulses 111a of the wavelength-tunable lasers 110a, each of which has a pulse width corresponding to 1/N of a repetition period of the plurality of wavelength-tunable lasers 110a.

As described above, according to example embodiments of the inventive concepts, the wavelength tuning speed can be increased by N times where ‘N’ corresponds to the number of the wavelength-tunable lasers 110, and the wavelength tuning bandwidth can be increased. Thus, accurate tomographic image information can be obtained.

In a conventional art, a wavelength-tunable laser has a limited driving speed, and thus an image faster than a wavelength tuning speed may not be obtained. However, according to the present embodiment, the wavelength tuning speed can be increased to correspond to the number of the wavelength-tunable lasers 110, thereby obtaining the accurate image.

The wavelength-tunable laser 110 according to the present embodiment may include any one of a Fourier domain mode locking laser based on a fiber Fabry-Perot filter, a Fourier domain mode locking laser based on a grating and a galvo mirror, a Fourier domain mode locking laser based on a grating and a polygon mirror, a fiber-based wavelength-tunable laser (e.g., a distributed control-based wavelength-tunable laser), a wavelength-tunable laser based on a polymer waveguide grating, and a wavelength-tunable laser based on MEMS VCSEL. However, embodiments of the inventive concepts are not limited thereto.

In addition, each of the plurality of wavelength-tunable lasers 110 may be connected to the optical coupling unit 120 through an optical waveguide 115. A core of the optical waveguide 115 becomes narrower in a guiding direction, and the narrowed core is connected to the optical coupling unit 120.

The optical waveguide 115 may be an optical fiber, a LiNbO₃ waveguide, an ion exchanged glass coupler, a SiO₂/Si waveguide, or a polymer waveguide. However, embodiments of the inventive concepts are not limited thereto.

The optical coupling unit 120 optically coupling the pulses sequentially provided from the plurality of wavelength-tunable lasers 110 may be any one of an array waveguide grating and a 1×N optical coupler. However, embodiments of the inventive concepts are not limited thereto.

As described above, according to the first embodiment of the inventive concepts, the tomography apparatus may rapidly increase the wavelength tuning speed by applying the interleaving technique to obtain the accurate tomographic image information, and thus the tomography apparatus can be widely used in medical fields (e.g., medical engineering and biomedical engineering), an aerospace field, a spectroscopy field, and a sensor field.

A low coherence interferometer-based tomography apparatus according to another embodiment of the inventive concepts will be described hereinafter. However, the descriptions to the same elements as in the first embodiment will be omitted in the following embodiment.

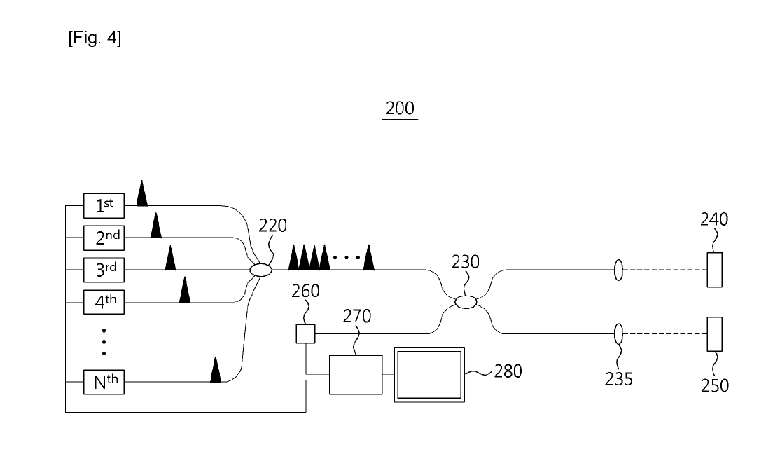

FIG. 4 is a schematic diagram illustrating a tomography apparatus based on a low coherence interferometer, according to a second embodiment of the inventive concepts.

As illustrated in FIG. 4, a low coherence interferometer-based tomography apparatus 200 according to a second embodiment of the inventive concepts may be similar to the tomography apparatus of the first embodiment. However, an element splitting pulses optically coupled by an optical coupling unit 220 in the present embodiment is different from a corresponding element of the first embodiment. An optical coupler 230 based on an optical waveguide is applied in the present embodiment.

In addition, pulses split by the optical coupler 230 may be provided to a sample stage 250 and a reference stage 240. At this time, a collimator 235 may be provided between the optical coupler 230 and each of the sample and reference stages 250 and 240 to generate parallel light. An optical detector 260 may obtain an interference signal from pulses transmitted through the optical coupler 230 via the sample stage 250 and the reference stage 240, and the obtained interference signal may be analyzed in a signal processing unit 170. Thereafter, signal-processed information may be displayed at a display unit 280 to obtain accurate tomographic image information.

A low coherence interferometer-based tomography apparatus according to a third embodiment of the inventive concepts will be described hereinafter. However, the descriptions to the same elements as in the aforementioned embodiments will be omitted in the third embodiment.

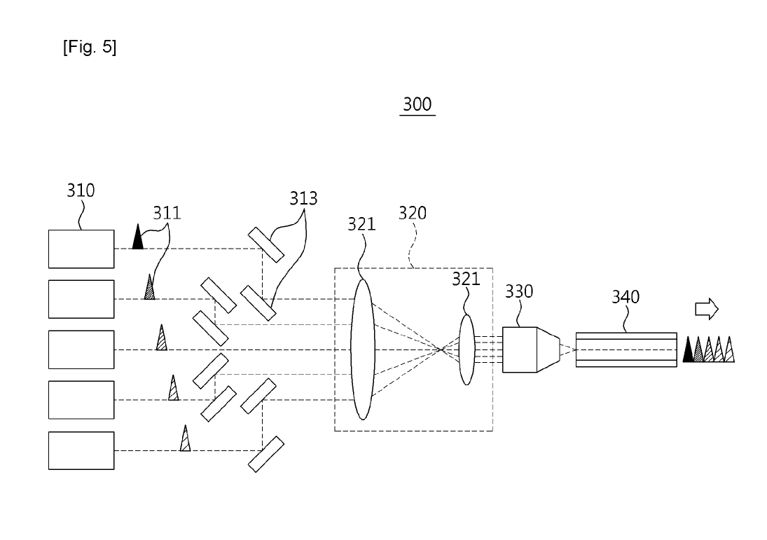

FIG. 5 is a schematic diagram illustrating a portion of a tomography apparatus based on a low coherence interferometer, according to a third embodiment of the inventive concepts.

As illustrated in FIG. 5, a low coherence interferometer-tomography apparatus 300 according to the third embodiment of the inventive concepts further includes a plurality of mirrors 313 respectively provided at the rears of a plurality of wavelength-tunable lasers 310 to parallel pulses 311 sequentially generated from the plurality of wavelength-tunable lasers 310, and a beam reduction unit 320 reducing a beam of pulses incident in parallel by the plurality of mirrors 313. By these elements, an optical coupling unit may be provided based on an optical waveguide 340 to optically couple the pulses beam-reduced by the beam reduction unit 320.

The pulses 311 are sequentially generated from the plurality of wavelength-tunable lasers 310. At this time, the pulses 311 generated from the wavelength-tunable lasers 310 may be incident in parallel to the beam reduction unit 320 by the mirrors 313 installed at the rears of the wavelength-tunable lasers 310.

The beam reduction unit 320 may include two lenses 321 and 325 and may beam-reduce the pulses 311 generated from the plurality of wavelength-tunable lasers 310 to transmit the beam-reduced pulses to a next object lens 330. The pulses transmitted through the object lens 330 may be transmitted to the optical coupling unit through the optical waveguide 340.

A low coherence interferometer-based tomography apparatus according to a fourth embodiment of the inventive concepts will be described hereinafter. However, the descriptions to the same elements as in the aforementioned embodiments will be omitted in the fourth embodiment.

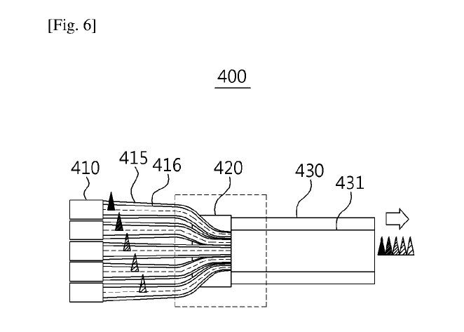

FIG. 6 is a schematic diagram illustrating a portion of a tomography apparatus based on a low coherence interferometer, according to a fourth embodiment of the inventive concepts.

As illustrated in FIG. 6, a plurality of wavelength-tunable lasers 410 included in a low coherence interferometer-based tomography apparatus 400 according to the present embodiment may be respectively connected to optical waveguides 415, each of which has a shape becoming narrower in a guiding direction.

An optical coupling unit 420 may be provided in an optical waveguide type having a core, and thus cores 416 of the optical waveguides 415 connected to the plurality of wavelength-tunable lasers 410 may be connected to a core 431 of an optical waveguide 430 connected to the optical coupling unit 420. As a result, pulses sequentially outputted from the plurality of wavelength-tunable lasers 410 may be interleaved and be coupled to each other.

A low coherence interferometer-based tomography apparatus according to a fifth embodiment of the inventive concepts will be described hereinafter. However, the descriptions to the same elements as in the aforementioned embodiments will be omitted in the fifth embodiment.

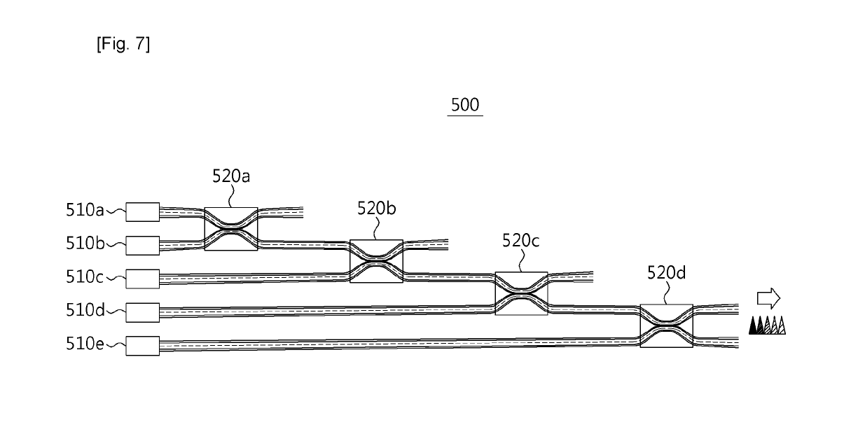

FIG. 7 is a schematic diagram illustrating a portion of a tomography apparatus based on a low coherence interferometer, according to a fifth embodiment of the inventive concepts.

As illustrated in FIG. 7, a low coherence interferometer-tomography apparatus 500 according to the present embodiment includes a plurality of optical coupling units 520a, 520b, 520c, and 520d.

Pulses sequentially generated from a first wavelength-tunable laser 510a and a second wavelength-tunable laser 510b of a plurality of wavelength-tunable lasers are optically coupled to each other by a first optical coupling unit 520a of the optical coupling units. In addition, a pulse generated by the first optical coupling unit 520a and a pulse generated from a third wavelength-tunable laser 510c of the plurality of wavelength-tunable lasers are optically coupled to each other by a second optical coupling unit 520b of the optical coupling units.

By this method, optical coupling processes can be sequentially performed up to the last wavelength-tunable laser 510e of the plurality of wavelength-tunable lasers 510a to 510e. At this time, the optical coupling by the interleaving technique is performed in each of the optical coupling units 520a, 520b, 520c, and 520d to increase the wavelength tuning speed.

According to embodiments of the inventive concepts, the tomography apparatus may rapidly increase the wavelength tuning speed by applying the interleaving technique to obtain the accurate tomographic image information, and thus the tomography apparatus can be widely used in medical fields (e.g., medical engineering and biomedical engineering), an aerospace field, a spectroscopy field, and a sensor field.

While the inventive concepts have been described with reference to exemplary embodiments, it will be apparent to those skilled in the art that various changes and modifications may be made without departing from the spirits and scopes of the inventive concepts. Therefore, it should be understood that the above embodiments are not limiting, but illustrative. Thus, the scopes of the inventive concepts are to be determined by the broadest permissible interpretation of the following claims and their equivalents, and shall not be restricted or limited by the foregoing description.

What is claimed is:

1. A tomography apparatus based on a low coherence interferometer, the tomography apparatus comprising:
a plurality of wavelength-tunable lasers arranged in parallel; and
an optical coupling unit interleaving pulses sequentially outputted from the plurality of wavelength-tunable lasers to increase a wavelength tuning speed of the wavelength-tunable lasers by N times where ‘N’ corresponds to the number of the wavelength-tunable lasers.

2. The tomography apparatus of claim 1, wherein the number of the plurality of wavelength-tunable lasers is N where ‘N’ is a natural number,
wherein the wavelength-tunable lasers have the same center wavelength and the same wavelength tuning range, and
wherein a speed of the wavelength-tunable lasers is increased by N times by interleaving and coupling the pulses of the plurality of wavelength-tunable lasers, each of which has a pulse width corresponding to 1/N of a repetition period of the plurality of wavelength-tunable lasers.

3. The tomography apparatus of claim 1, wherein the number of the plurality of wavelength-tunable lasers is N where ‘N’ is a natural number,
wherein center wavelengths and wavelength tuning ranges of the wavelength-tunable lasers sequentially increase, and
wherein a wavelength tuning bandwidth N times wider than the maximum wavelength tuning bandwidth of the wavelength-tunable lasers is obtained by interleaving and coupling the pulses of the plurality of wavelength-tunable lasers, each of which has a pulse width corresponding to 1/N of a repetition period of the plurality of wavelength-tunable lasers.

4. The tomography apparatus of claim 1, further comprising:
a splitting unit connected to the optical coupling unit to split pulses optically coupled by the optical coupling unit into a sample stage and a reference stage; and
an optical detector obtaining an interference signal from pulses transmitted through the splitting unit via the sample stage and the reference stage.

5. The tomography apparatus of claim 4, wherein the splitting unit is a beam splitter splitting a beam or an optical coupler based on an optical waveguide.

6. The tomography apparatus of claim 1, further comprising:
a plurality of mirrors respectively provided at rears of the plurality of wavelength-tunable lasers to parallel the pulses generated from the plurality of wavelength-tunable lasers; and
a beam reduction unit reducing a beam of the pulses incident in parallel by the plurality of mirrors,
wherein the optical coupling unit is provided based on an optical waveguide to optically couple the pulses beam-reduced by the beam reduction unit.

7. The tomography apparatus of claim 1, wherein the plurality of wavelength-tunable lasers are respectively connected to optical waveguides, each of which has a shape becoming narrower in a guiding direction, and
wherein the optical coupling unit is provided in an optical waveguide type having a core such that cores of the optical waveguides connected to the plurality of wavelength-tunable lasers are connected to the core of the optical coupling unit to interleave the pulses sequentially outputted from the plurality of wavelength-tunable lasers.

8. The tomography apparatus of claim 1, wherein the optical coupling unit is provided in plurality, and
wherein: the pulses sequentially generated from a first wavelength-tunable laser and a second wavelength-tunable laser of the plurality of wavelength-tunable lasers are optically coupled to each other by a first optical coupling unit of the optical coupling units; a pulse generated by the first optical coupling unit and the pulse generated from a third wavelength-tunable laser of the plurality of wavelength-tunable lasers are optically coupled to each other by a second optical coupling unit of the optical coupling units; and optical coupling processes are sequentially performed up to the last wavelength-tunable laser of the wavelength-tunable lasers by the optical coupling method.

9. The tomography apparatus of claim 1, wherein the optical coupling unit is any one of an array waveguide grating and a 1×N optical coupler.

10. The tomography apparatus of claim 1, wherein each of the plurality of wavelength-tunable lasers is connected to the optical coupling unit through an optical waveguide, and
wherein the optical waveguide is any one of an optical fiber, a LiNbO₃ waveguide, an ion exchanged glass coupler, a SiO₂/Si waveguide, and a polymer waveguide.

11. The tomography apparatus of claim 1, wherein each of the wavelength-tunable lasers is any one of a Fourier domain mode locking laser based on a fiber Fabry-Perot filter, a Fourier domain mode locking laser based on a grating and a galvo mirror, a Fourier domain mode locking laser based on a grating and a polygon mirror, a fiber-based wavelength-tunable laser of a distributed control-based wavelength-tunable laser, a wavelength-tunable laser based on a polymer waveguide grating, and a wavelength-tunable laser based on MEMS VCSEL.
